(19) United States
(12) Patent Application Publication
(10) Pub. No.: US 2009/0247911 A1
(43) Pub. Date: Oct. 1, 2009

(54) Multiple-angle switched high intensity focused ultrasound

(76) Inventors: Petr Novak, Pardubice (CZ); Robert J. Griffin, Little Rock, AR (US)

Correspondence Address: WRIGHT, LINDSEY & JENNINGS LLP, 200 WEST CAPITOL AVENUE, SUITE 2300, LITTLE ROCK, AR, 72201-3699, US

(21) Appl. No.: 12/383,436

(22) Filed: Mar. 24, 2009

Related U.S. Application Data
(60) Provisional application No. 61/070,725, filed on Mar. 25, 2008.

(51) Int. Cl. A61N 7/00

(52) U.S. Cl. 601/2

(57) ABSTRACT

A multiple-angle switched high intensity focused ultrasound device having at least two focused ultrasound transducers mounted in a frame so that the focal zones of all transducers intersect. Each of the transducers is independently driven from a radiofrequency (RF) generator. The RF driving signal is switched so that no more than one transducer is operated at a time. This allows shaping the temperature distribution in the target area by overlapping thermal contributions of each transducer.

### CROSS-REFERENCE TO RELATED APPLICATIONS

This application claims the benefit of U.S. Provisional Patent Application No. 61/070,725 filed Mar. 25, 2008, the disclosure of which is incorporated herein by reference in its entirety.

### STATEMENT REGARDING FEDERALLY SPONSORED RESEARCH OR DEVELOPMENT

Not applicable.

### BACKGROUND OF THE INVENTION

1. Field of the Invention

The present invention relates to ultrasound devices for delivering ultrasound energy (which may or may not result in temperature increases in target tissues) to target tissues, and in particular, to such a device having multiple transducers.

2. Brief Description of the Related Art

Multiple ultrasound transducers have been used to create a focal zone of sufficient power and of shape suitable for thermal therapies. These devices drive all or at least two of the transducers at the same time to deliver sufficient power to the target zone. The focal zone that results is the result of acoustical contributions of the ultrasound transducers. These contributions are difficult to predict when considering real tissue as it depends on mutual directions and phase shifts of contributing acoustical waves at a sub-millimeter level coming from individual ultrasound transducers. The current devices are unable to create small (<1 cm) spherical lesions. When these devices are used for thermal ablation, they typically ablate tissue by performing several individual ablations in series. Each of the individual ablations creates a “cigar-shaped” lesion by the nature of the way ultrasound energy is focused in these devices. If a spherical volume of tissue smaller than the individual ablation needs to be ablated while sparing surrounding tissue, current ultrasound devices are unable to do that. In addition, creating multiple cigar-like lesions promotes a tendency to miss tissue and allow regrowth or recurrence of the disease in question.

The limitations of the prior art are overcome by the present invention as described below.

### BRIEF SUMMARY OF THE INVENTION

The present invention is a multiple-angle switched high intensity focused ultrasound device. The device is for (1) delivering thermal therapy, (2) triggering heat-activated therapies/drug/gene delivery, and (3) triggering ultrasound-activated therapies/drug/gene delivery. The device consists of at least two focused ultrasound transducers, wherein each of the transducers are mounted in a frame at different angles so that the focal zones of all transducers intersect at a selected target focal zone. A focused array of transducers that are switched on and off as a unit are considered herein to be a single transducer. Each of the transducers is independently driven from a radiofrequency (RF) generator. The RF driving signal is designed so that only one transducer is operated at a time (i.e. the power is switched from one transducer to the other in various temporal patterns). This allows shaping the temperature distribution in the target area by overlapping thermal contributions of each transducer. The design of the driving signal also allows for including periods of time into the operation of the device when no transducer is driven in order to let the heat transfer properties of tissue, such as blood perfusion, influence the temperature distribution as well as to use various imaging/treatment techniques without any interference with the ultrasound system.

The device of the present invention is conceptualized so that the acoustical pressure distribution in the focal zone is identical to the use of a single transducer at any time (there is at most one transducer energized at any time), which is much easier to model and predict-even in real tissue-than for current devices, and the resulting temperature distribution (if used for thermal therapy or thermally activated/triggered therapy) is therefore much less dependent on the alignment of the ultrasound transducers as well as scattering properties of tissue. The present invention when used as a thermal therapy (ablation and/or hyperthermia) system has the ability to easily deliver highly therapeutic heat doses to the center of malignant tumors or other tissue regions, thereby enhancing the effect of other therapies applied in combination to completely sterilize the tumor. These therapies are envisioned as leading edge methods of radiation therapy and/or targeted chemotherapy.

The present invention may be used for (1) delivery of thermal ablation to induce well-defined, predictable, sphere-like lesions; (2) delivery of hyperthermia to small well-defined locations in human disease and veterinary medicine applications as well as animal-based research; and (3) acoustical triggering of other therapies.

These and other features, objects and advantages of the present invention will become better understood from a consideration of the following detailed description of the preferred embodiments and appended claim in conjunction with the drawings as described following:

### BRIEF DESCRIPTION OF THE SEVERAL VIEWS OF THE DRAWINGS

FIG. 1 is a block diagram of the hardware components of the present invention.

FIG. 2 is a perspective view of an embodiment of the present invention showing two transducers mounted at a 90° angle to one another and including a coupling bolus. The lines are laser beams allowing visualization of ultrasound beams intersecting at a target focal zone.

### DETAILED DESCRIPTION OF THE INVENTION

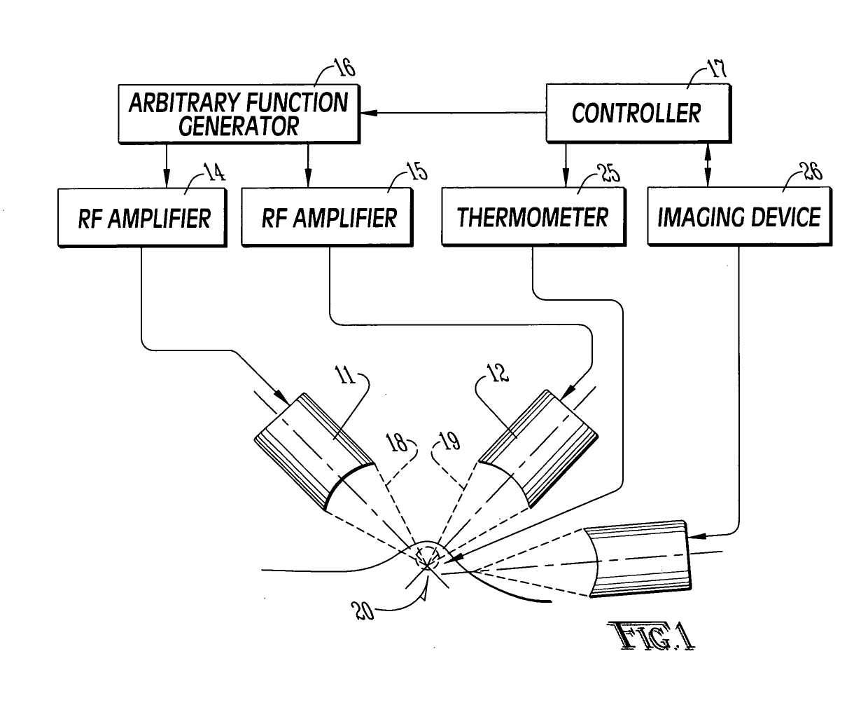

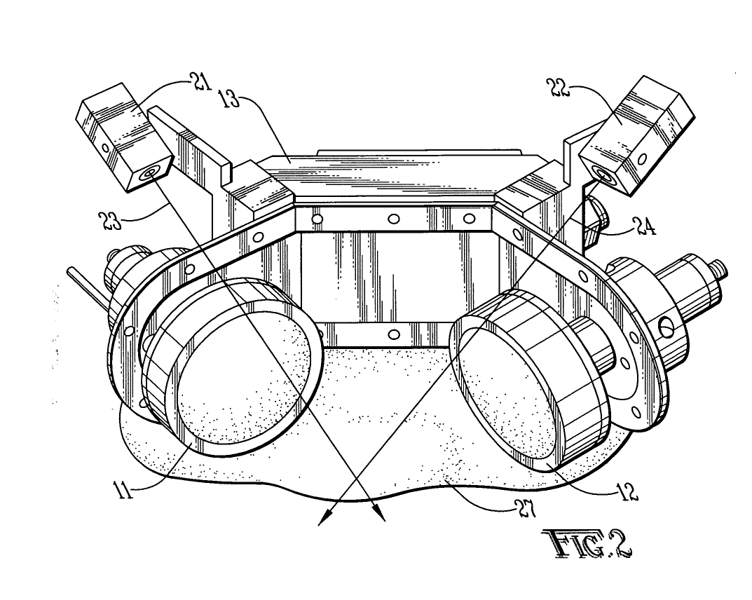

With reference to FIGS. 1 and 2, the preferred embodiment of the present invention may be described.

The present invention is a multiple-angle switched high intensity focused ultrasound device. The device is for (1) delivering thermal therapy, (2) triggering heat-activated therapies/drug/gene delivery, and (3) triggering ultrasound-activated therapies/drug/gene delivery. As shown in the embodiment of FIGS. 1 and 2, the device consists of at least two focused ultrasound transducers 11, 12 mounted at separate angles in a frame 13 so that the focal zones of all transducers intersect at a target focal zone 20. FIG. 2 shows two transducers 11, 12 mounted at a 90° angle. Laser pointers 21, 22 with respective beams 23, 24 allow the center of the target focal zone 20 to be visualized as the intersection of the beams 23, 24. As shown in FIG. 1, the ultrasound beams 18, 19 from the transducers 11, 12 intersect at the target focal zone 20. A focused array of transducers that are switched on and off as a unit are considered herein to be a single transducer. Thus each of the transducers 11, 12 may be in the form of an array of individual transducers focused on the same zone and switched on and off as a unit.

As shown in the embodiment of FIG. 1, each of the transducers 11, 12 is independently driven from a radiofrequency (RF) generator, which comprises separate RF amplifiers 14, 15 for each transducer 11, 12, respectively, and an arbitrary function generator 16 driving the RF amplifiers 14, 15. The arbitrary function generator 16 is controlled by a controller 17, e.g., a PC, such that the RF driving signal to each transducer 11, 12 is switched so that no more than one transducer 11, 12 is operated at a time (i.e., the power is switched from one transducer to the other in various temporal patterns). This allows shaping of the temperature distribution in the target focal zone 20 by overlapping the thermal contribution from each beam 18, 19 of each transducer 11, 12, respectively. The design of the driving signal also allows for including periods of time into the operation of the device, where no transducer is driven in order to let the heat transfer properties of the tissue, such as blood perfusion, influence the temperature distribution as well as to use various imaging/treatment techniques without any interference with the ultrasound system. Imaging may be provided by imaging device 26, which may be any of various types of imaging devices known to those skilled in the art, such as ultrasound imaging. Temperature feedback may be provided to the controller 17 by a temperature sensing device, such as thermometer 25. This embodiment illustrates only one possible embodiment of the present invention. Various modifications would be apparent to one of skill in the art. For example, the same functions could be performed by multiple generators or with a multi-channel device that combines the functions of generator and amplifier. A thin flexible membrane 27 is attached to the frame 13 so it encloses the space around the transducers 11 and 12. The membrane is large enough so that the entire volume between the transducers and the surface of the treated object can be filled with water providing appropriate ultrasound coupling for the ultrasound energy traveling from the transducers 11 and 12 to the target tissue. This is known as a “coupling bolus” to those skilled in art.

The device of the present invention is conceptualized so that the acoustical pressure distribution in the focal zone 20 is identical to the use of a single transducer at any time (there is at most one transducer energized at any time), which is much easier to model and predict—even in real tissue—than for current devices. The temperature distribution generated by the device (if used for thermal therapy or thermally activated/triggered therapy) is also much easier to predict, because there is no influence of phase shift of the ultrasound signal on the temperature distribution and the resulting temperature distribution is therefore much less dependent on the alignment of the ultrasound transducers as well as scattering properties of tissue. The device is also able to easily deliver highly therapeutic heat doses to the center of malignant tumors or other tissue regions, thereby enhancing the effect of other therapies applied in combination to completely sterilize the tumor and avoiding tissue damage typical with invasive heating strategies. These therapies are envisioned as leading edge methods of radiation therapy and/or targeted chemotherapy.

The device may be used for (1) delivery of thermal ablation to induce well-defined, predictable, sphere-like lesions; (2) delivery of hyperthermia to small well-defined locations in human disease and veterinary medicine applications, as well as animal-based research research; and (3) acoustical triggering of other therapies.

High intensity focused ultrasound (HIFU), when used for noninvasive thermal ablation of localized tumors, has a cigar-like shape, while tumors are often spherical. The HIFU is therefore used for larger target volumes where a series of ablations can be delivered to conform to a tumor. The present invention, however, non-invasively creates sphere-like lesions, which is especially beneficial for—but not restricted to—small or medium lesions. This approach may benefit both clinical thermal ablation in humans as well as in small-animal research. Varying the incident angles of acoustical beams and power/timing configurations allow creating energy distributions which are not restricted to sphere-like shapes only.

In one embodiment, the ultrasound ablation device of the present invention consists of 2 spherically-focused ultrasound transducers (f=2.25 MHz, d=38 mm, sf=51 mm) with focal zones crossing each other at a 90 degree angle. The arrangement assures maximum energy deposition to the overlapping volume of the focal zones. The transducers are alternated in their operation to provide time for heat dissipation from areas outside of the overlapping volume. The procedure was modeled for a homogeneous media with acoustical and heat transfer parameters typical for muscle tissue for blood perfusions of 0, 3, 6, and 9 kg/m3 s. The transducers were simulated at 40 W acoustical powers with a 17% duty cycle (250 ms on, 1250 ms off) and a mutual delay of 750 ms.

The simulated lesions had a roughly spherical shape corresponding to the overlapping volume of the ultrasound focal zones. The diameter of ablated region (CEM43C>240) was 1.9 mm, 3.0 mm, 3.9 mm, 5.0 mm, and 11.2 mm for ablation durations of 5 s, 6 s, 7 s, 10 s, and 25 s, respectively. Neither the size nor the shape of lesions was significantly affected by simulated blood perfusion.

The approach of the present invention represents a simple and relatively inexpensive means for noninvasive delivery of thermal ablation to small sphere-like volumes within several seconds. The ability to allow imagine routines to be run between HIFU cycles is a prominent feature of the device which improves significantly on much of the current art in the field of thermal ablation.

The present invention has been described with reference to certain preferred and alternative embodiments that are intended to be exemplary only and not limiting to the full scope of the present invention.

What is claimed is:

1. A multiple-angle switched high intensity focused ultrasound device, comprising:
a frame;
at least two focused ultrasound transducers mounted in said frame such that the focal zones of all of said transducers intersect;
a radiofrequency (RF) generator providing an RF driving signal to each of said transducers so that no more than one of said transducers is operated at a time.

2. The device of claim 1, wherein said radiofrequecy generator comprises at least two RF amplifiers, each of said RF amplifiers providing an RF driving signal to one of said transducers;
a function generator driving said RF amplifiers; and
a controller controlling said function generator.

3. The device of claim 1, further comprising a coupling bolus.

4. The device of claim 1, further comprising a temperature sensing device.

5. The device of claim 1, further comprising an imaging device.

6. The device of claim 1, wherein said transducers comprise spherically-focused transducers.

7. The device of claim 1, wherein said transducers are disposed such that the focal zones of each transducer cross at 90 degrees to the focal zones of the other transducers.

8. The device of claim 1, wherein said radiofrequency generator operates said driving signals so that periods of time occur when no transducer is operating.
